(19) United States
(12) Patent Application Publication
(10) Pub. No.: US 2019/0183890 A1
(43) Pub. Date: Jun. 20, 2019

(54) METHODS AND COMPOSITIONS FOR PROMOTING HAIR GROWTH

(71) Applicant: Rophe Pharma, Inc., Burbank, CA (US)

(72) Inventors: Robert GADIMIAN, Burbank, CA (US); Donald Place, Montrose, CA (US)

(21) Appl. No.: 16/322,939

(22) PCT Filed: Aug. 3, 2017
(86) PCT No.: PCT/US17/45317

§ 371 Date: Feb. 2, 2019

Related U.S. Application Data
(60) Provisional application No. 62/370,683, filed on Aug. 3, 2016.

(51) Int. Cl. A61K 31/502; A61P 17/14; A61K 47/10; A61K 9/00; A61K 31/58; A61K 31/575; A61K 31/192

(52) U.S. Cl. CPC A61K 31/502; A61P 17/14; A61K 47/10; A61K 31/192; A61K 31/58; A61K 9/0053; A61K 31/575; A61K 9/0014

(57) ABSTRACT

Disclosed are methods and compositions for treating alopecia in a subject. The methods and compositions include hydralazine in a pharmaceutically acceptable preparation for topical administration. Also disclosed are combinations of hydralazine and a type II 5α-reductase, an anti-inflammatory agent, or a vasoconstrictor.

### CROSS-REFERENCE TO RELATED APPLICATIONS

This application is related to, and claims the benefit of, U.S. Provisional Application No. 62/370,683, filed Aug. 3, 2016. The above-identified priority patent applications is incorporated herein by reference in its entirety.

### FIELD OF INVENTION

The present invention relates to methods and compositions for promoting hair growth and, in particular, to topical methods and compositions for treating alopecia.

### INTRODUCTION

Hair loss disorders represent a common problem affecting both men and women. Androgenic alopecia or male pattern baldness is the most common of such disorders (Gupta, M. et al., Classifications of Patterned Hair Loss: A Review, J Cutan Aesthet Surg. 9(1): 3-12 (2016)).

Topical minoxidil and oral finasteride are the only drugs approved by the FDA for treatment of male pattern baldness (Mounsey, A. L. et al., Diagnosing and treating hair loss, Am Fam Physician, 80(4):356-62 (2009)). While finasteride is a competitive and specific inhibitor of Type II 5α-reductase, the mechanism of action of minoxidil is not clearly understood (Cranwell W and Sinclair R., In: De Groot L. J. et al, editors. Endotext [Internet]. South Dartmouth (MA): MDText.com, Inc.; 2000-2016 Feb. 29). A number of possible mechanisms of action have been proposed for minoxidil including vasodilation resulting in an increase in microcirculation surrounding the hair follicle. (Wester R. C, et al., Minoxidil stimulates cutaneous blood flow in human balding scalps: pharmacodynamics measured by laser Doppler velocimetry and photopulse plethysmography. The Journal of investigative dermatology, 82(5):515-517 (1984)). Nevertheless, vasodilators have not been generally known to effect treatment of alopecia and none other than minoxidil have been approved by the FDA.

### SUMMARY

Accordingly, the present inventors have succeeded in discovering that the vasodilator, hydralazine, alone or in combination with other agents may serve as an effective topical treatment of alopecia.

Thus, in various embodiments, the present invention may include a method of treating alopecia in a subject. The method includes topically administering to a subject in need thereof, an effective amount of hydralazine in a pharmaceutically acceptable preparation.

In various embodiments, the present invention may also include a topical composition for treating alopecia comprising an effective amount of hydralazine in a pharmaceutically acceptable preparation.

In various of the above embodiments, the hydralazine may be present in an amount of from about 0.1% to about 4% and, in particular, in an amount of about 2%. The pharmaceutically acceptable preparation may be an aqueous preparation the includes a glycol such as ethylene glycol or propylene glycol and an alcohol such as methanol, ethanol, isopropanol, or n-propanol. In various embodiments, the hydrazine may be hydralazine HCl and the pharmaceutically acceptable aqueous preparation may include about 25% propylene glycol and about 50% ethanol. Further, the preparation may be suitable for twice daily topical administration.

In various other embodiments, the methods and compositions may further include an agent selected from the group consisting of a type II 5α-reductase, an anti-inflammatory agent, and a vasoconstrictor. The type II 5α-reductase may be finasteride administered orally or topically; the anti-inflammatory agent may be a steroidal anti-inflammatory agent such as a corticosteroid or non-steroidal anti-inflammatory agent such as ibuprofen; and the vasoconstrictor may be betamethasone dipropionate.

### DETAILED DESCRIPTION

The present invention is directed to methods and compositions for treating alopecia in a subject. The methods and compositions include hydralazine in a pharmaceutically acceptable preparation for topical administration

### Definitions

As used herein, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

As used herein, the term “about” is intended to refer to a range of values above and below a stated value such as for example, values encompassing 10% below up to 10% above a stated value.

The term “and/or” is intended to mean either or both of two recited elements.

The term “inhibitor” refers to a substance that can reduce or prevent the activity of an enzyme or enzyme system. For example, an inhibitor of 5α-reductase reduces, diminishes or prevents the activity of the enzyme.

The terms “substance”, “agent” or “compound” may be used interchangeably herein in connection with treating a disease or condition. The substances, agents or compounds of the present invention may be an active pharmaceutical ingredient (API) in a pharmaceutically acceptable formulation.

Reference herein to an API may include hydralazine compounds such as hydralazine and its derivatives, analogues and the like as well as pharmaceutically acceptable solvates, salts, hydrates or hydrated salts, their optical isomers, racemates, diastereomers, enantiomers or the polymorphic crystalline structures of the compounds. An API may also refer to a concomitantly administered drug including a type II 5α-reductase such as finasteride, a steroidal anti-inflammatory agent such as a corticosteroid, a non-steroidal anti-inflammatory agent such as ibuprofen or a vasoconstrictor such as betamethasone dipropionate.

The term “pharmaceutical composition” or “pharmaceutically acceptable composition” and a “pharmaceutical preparation” or “pharmaceutically acceptable preparation” refer to a composition or preparation that combines one or more API's with a pharmaceutically acceptable carrier such that the composition or preparation is suitable for therapeutic use in vitro, in vivo or ex vivo.

As used herein, the term “pharmaceutically acceptable carrier” encompasses any suitable pharmaceutical carriers, such as a phosphate buffered saline solution, water, and emulsions, such as an oil/water or water/oil emulsion, various types of wetting agents and the like. The compositions also can include stabilizers and preservatives. Examples of carriers, stabilizers and adjuvants, can be found in Remington: The Science and Practice of Pharmacy, Lippincott Williams & Wilkins, Twenty-First edition (May 19, 2005).

Reference herein to an amount of a substance in a formulation such as a topical formulation may be given in terms of percent weight/volume (w/v), i.e. in terms of grams/100 ml.

The terms “treatment” or “treating” as used herein, may include ameliorating, suppressing, eradicating, reducing the severity of, decreasing the frequency of incidence of, preventing, reducing the risk of, and/or delaying the onset of a disease or condition. In various embodiments, the treatment may be targeted to the underlying disease and not to disease symptoms or to ancillary pathologic processes that are not directly related to the underlying disease. In various other embodiments, the treatment may target the underlying disease, disease symptoms and ancillary pathological processes or any combination thereof.

The terms “concomitant administration” or “administration of a combination” is intended to mean simultaneous or sequential administration of two or more agents. These may be administered in any order or they may be administered together in one topical composition.

The terms “androgenic alopecia”, “androgenetic alopecia” and “male pattern baldness” may be used interchangeably herein when referring to hair loss in either of the sexes after puberty, typically presenting with progressive thinning, miniaturization, and loss of hair on the scalp (Gupta, M. et al., Classifications of Patterned Hair Loss: A Review, J Cutan Aesthet Surg. 9(1): 3-12 (2016)).

The term “subject” or “patient” as used herein typically denotes humans, but may also encompass reference to non-human animals, in particular warm-blooded animals, and even more particularly mammals, such as, e.g., non-human primates, rodents, canines, felines, equines, ovines, porcines, and the like. Further, subjects may include both male and female genders.

Hydralazine Compounds

Reference to hydralazine compounds includes hydralazine and its derivatives, analogues and the like and these may include salts such as the hydrochloride salt thereof. For example, hydralazine may be in the form of a hydrochloride salt having the structure:

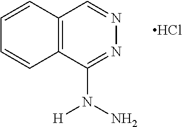 [SMILES: NNc1nncc2ccccc12 ; Cl]

The chemical name of hydralazine is 1-hydrazinophthalazine monohydrochloride. Examples of hydralazine compounds that are derivatives or analogues of hydralazine include budralazine, cadralazine, dihydralazine, endralazine, hydralazine, pildralazine, todralazine and the like.

Hydralazine or other hydralazine compound may be present in a topical formulation of the present invention in an amount of from about 1% (w/v), about 2% (w/v), about 3% (w/v), about 4% (w/v), about 5% (w/v), about 6% (w/v), about 7% (w/v), about 8% (w/v), about 9% (w/v), about 10% (w/v) up to about 11% (w/v), about 12% (w/v), about 13% (w/v), about 14% (w/v), about 15% (w/v), about 16% (w/v), about 17% (w/v), about 18% (w/v), about 19% (w/v) or about 20% (w/v) and, in particular, about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), about 11% (w/v), about 12% (w/v), about 13% (w/v), about 14% (w/v), about 15% (w/v), about 16% (w/v), about 17% (w/v), about 18% (w/v), about 19% (w/v) or about 20% (w/v).

The topical formulation of hydralazine or other hydralazine compound may include one or more pharmaceutically acceptable carrier substances including, but not limited to, saline, aqueous electrolyte solutions, an alcohol such as methanol, methanol, ethanol, isopropanol or n-propanol, dimethyl sulfoxide, dimethyl isosorbide, isopropyl myristate, lauryl lactate, diisopropyl adipate, sodium lauryl sulfoacetate; ionic and nonionic osmotic agents such as sodium chloride, potassium chloride, glycerol, a glycol such as ethylene glycol or propylene glycol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfate, ascorbic acid, acetyl cysteine, cysteine, glutathione, butylated hydroxyanisole, butylated hydroxytoluene, tocopherols, and ascorbyl palmitate; compounds such as lecithin, phospholipids; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate. Such pharmaceutically acceptable carriers may be preserved against bacterial contamination using preservatives, including, but not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

Administration may be once a day (q.d.), twice a day (b.i.d.), three times a day (t.i.d.), four times a day (q.i.d.) or at more or less frequent intervals such as once every other day (q.a.d.), once every third day, twice a week (bis in 7 d.), once a week (QWK), once every other week, etc. Alternatively, administration may be as needed (p.r.n.).

Combinations with Hydralazine

Hydralazine and Finasteride

Finasteride, is a type II 5α-reductase that acts by reducing dihydrotestosterone levels in patients with androgenetic alopecia (male pattern baldness). As noted above, the FDA has approved finasteride for the treatment of androgenic alopecia. A topical formulation of 5% minoxidil and 0.1% finasteride has been shown to be an effective treatment of in patients with androgenic alopecia after initial treatment with 5% topical minoxidil and oral finasteride for two years. (Chandrashekar, B. S. et al., Topical minoxidil fortified with finasteride: An account of maintenance of hair density after replacing oral finasteride, Indian Dermatol Online J. 6(1):17-20 (2015)).

Thus, the combination of hydralazine and finasteride would also be expected to show an improvement in hair regrowth in subjects with androgenetic alopecia in view of the discovery herein of the beneficial effect of hydralazine alone. Accordingly, the treatment with topical hydralazine and oral finasteride as well as hydralazine and finasteride together in a topical formulation may be used in subjects with androgenetic alopecia.

Topical hydralazine along with oral finasteride may be administered in topical amounts of hydralazine as noted above (about 1% (w/v) to about 10% (w/v)) and oral finasteride may be administered in amounts of about 0.25 mg, about 0.5 mg, about 0.75 mg, about 1.0 mg, about 1.25 mg, about 1.5 mg, about 1.75 mg or about 2.0 mg and in particular, about 1.0 mg.

A topical formulation including both hydralazine and finasteride may include hydralazine in an amount as noted above (about 1% (w/v) to about 10% (w/v)) and finasteride may be included in an amount of about 0.5% (w/v), about 1.0% (w/v), about 1.5% (w/v), about 2.0% (w/v), about 2.5% (w/v), about 3.0% (w/v), about 3.5% (w/v), about 4.0% (w/v), about 4.5% (w/v) or about 5.0% (w/v).

The topical formulation may also include a pharmaceutically acceptable carrier system as described above.

Hydralazine and an Anti-Inflammatory Agent

The beneficial effect of a combination of minoxidil and an anti-inflammatory agent has been reported (Kligman, A. M., WO8807361). The anti-inflammatory agent may be steroidal agent such as a corticosteroid or non-steroidal agent such as ibuprofen. Steroidal compounds were used at a concentration of 0.4 to 2.5% and non-steroidal agents were used at a concentration of from 1 to 5%. They may be administered orally or topically in combination with minoxidil administered topically in an amount of 1 to 10%.

Thus, the combination of hydralazine and an anti-inflammatory agent would also be expected to show an improvement in hair regrowth in subjects with androgenetic alopecia in view of the discovery herein of the beneficial effect of hydralazine alone. Accordingly, the treatment with topical hydralazine and an oral anti-inflammatory agent as well as hydralazine and an anti-inflammatory agent together in a topical formulation may be used in subjects with androgenetic alopecia.

Topical hydralazine along with an oral anti-inflammatory agent may be administered in topical amounts of hydralazine as noted above (about 1% (w/v) to about 10% (w/v)) and an oral steroidal anti-inflammatory agent may be administered in an amount of about 0.25 mg, about 0.5 mg, about 0.75 mg, about 1.0 mg, about 1.25 mg, about 1.5 mg, about 1.75 mg, about 2.0 mg, about 3.0 mg, about 4.0 mg, about 5.0 mg, about 6.0 mg, about 7.0 mg, about 8.0 mg, about 9.0 mg, about 10.0 mg, about 11.0 mg or about 12.0 mg and/or an oral non-steroidal anti-inflammatory agent may be administered in an amount of about 100 mg, about 200 mg., about 300 mg, about 400 mg, about 500 mg, about 600 mg, about 700 mg, about 800 mg, about 900 mg or about 1000 mg.

A topical formulation including both hydralazine and an anti-inflammatory agent may include hydralazine in an amount as noted above (about 1% (w/v) to about 10% (w/v)) and a steroidal anti-inflammatory agent may be included in an amount of about 0.2% (w/v), about 0.4% (w/v), about 0.6% (w/v), about 0.8% (w/v), about 1.0% (w/v), about 1.5% (w/v), about 2.0% (w/v) or about 2.5% (w/v) and/or a non-steroidal anti-inflammatory agent may be included in an amount of about 0.5% (w/v), about 1.0% (w/v), about 2.0% (w/v), about 3.0% (w/v), about 4.0% (w/v) or about 5.0% (w/v).

The topical formulation may also include a pharmaceutically acceptable carrier system as described above.

Hydralazine and Vasoconstrictors

The beneficial effect of a combination of minoxidil and a vasoconstrictor agent has been reported (Fielder, V. C. EP0451156). The amount of minoxidil was at least 2.5%. The vasoconstrictor may be a corticosteroid or a scopolamine, e.g. betamethasone dipropionate. The amount of vasoconstrictor was from about 0.01 to about 1.0%

Thus, the combination of hydralazine and a vasoconstrictor agent would also be expected to show an improvement in hair regrowth in subjects with androgenetic alopecia in view of the discovery herein of the beneficial effect of hydralazine alone. Accordingly, the treatment with hydralazine and a vasoconstrictor agent together in a topical formulation may be used in subjects with androgenetic alopecia.

A topical formulation including both hydralazine and a vasoconstrictor agent may include hydralazine in an amount as noted above (about 1% (w/v) to about 10% (w/v)) and a vasoconstrictor agent such as betamethasone dipropionate in an amount of about 0.01% (w/w), about 0.05% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v) or about 1.0% (w/v).

The topical formulation may also include a pharmaceutically acceptable carrier system as described above.

Other Combinations

Kingman, A M (U.S. Pat. No. 5,026,691) cites Japanese patent Kokai 61-260010 as stating that “topical minoxidil formulations containing. e.g., amino acids, bactericides, anti-inflammatory agents, adrenal hormones, antihistaminics, vitamin E derivatives, estrogens, and capillary vessel dilators may be prepared.”

Thus, the combination of hydralazine and an agent such as amino acid, a bactericide, an adrenal hormone, an antihistaminic, a vitamin E derivative, an estrogen, or a capillary vessel dilator would also be expected to show an improvement in hair regrowth in subjects with androgenetic alopecia in view of the discovery herein of the beneficial effect of hydralazine alone. Accordingly, the treatment with hydralazine and an agent such as amino acid, a bactericide, an adrenal hormone, an antihistaminic, a vitamin E derivative, an estrogen, or a capillary vessel dilator, together in a topical formulation may be used in subjects with androgenetic alopecia.

### Examples

This example illustrates the treating of patients with hydralazine for androgenic alopecia.

1) A 0.2% solution of hydralazine was prepared in glycol, alcohol and water and applied to over 200 patients who mostly has androgenic alopecia. The hair grew back and the hair growth was visible after the first month. However, the treatment has to be continued every month to sustain the hair that has grown.

2) Hydralazine has been tested on 12 men aging from 33-69 years to date. The starting dose was 150 mg/100 ml. Since no side effect was observed, the dose was increased first to 300 mg/100 ml and then to 600 mg/100 ml. Visible enhancement in the hair count and hair thickness has been observed in the 11 out of the 12 men after 6-8 weeks of treatment which is much faster onset of effect than Rogaine. No side effect is reported by anybody even with the 600 mg/ml dose thus we intend to increase the dose to 1 g/100 ml and eventually to 2 g/100 ml with is the original approved Rogaine (minoxidil) dose.

As various changes could be made in the above methods and compositions without departing from the scope of the invention, it is intended that all matter contained in the above description shall be interpreted as illustrative and not in a limiting sense.

All references cited in this specification, including patents and patent applications, are hereby incorporated by reference. The discussion of references herein is intended merely to summarize the assertions made by their authors and no admission is made that any reference constitutes prior art. Applicant reserves the right to challenge the accuracy and pertinence of the cited references.

What is claimed is:

1. A method of treating alopecia in a subject, the method comprising topically administering to a subject in need thereof, an effective amount of hydralazine in a pharmaceutically acceptable preparation.

2. The method of claim 1, wherein the hydralazine is present in an amount of from about 0.5% to about 4%.

3. The method of claim 2, wherein the hydralazine is present in the preparation in an amount of about 2%.

4. The method of claim 1, wherein the hydralazine is in a pharmaceutically acceptable aqueous preparation further comprising a glycol and an alcohol.

5. The method of claim 4, wherein the glycol is ethylene glycol or propylene glycol

6. The method of claim 4, wherein the alcohol is a monohydroxy alcohol selected from the group consisting of methanol, methanol, ethanol, isopropanol, and n-propanol.

7. The method of claim 4, wherein the hydralazine is hydralazine HCl and the pharmaceutically acceptable aqueous preparation comprises about 25% propylene glycol and about 50% ethanol or only purified water.

8. The method of claim 1, wherein the method comprises twice daily topical administration.

9. The method of claim 1, wherein the method further comprises administering an agent selected from the group consisting of a type II 5α-reductase, an anti-inflammatory agent, and a vasoconstrictor.

10. The method of claim 9, wherein the a type II 5α-reductase is finasteride administered orally.

11. The method of claim 9, wherein the anti-inflammatory agent is a steroidal or non-steroidal anti-inflammatory agent.

12. The method of claim 11, wherein the steroidal anti-inflammatory agent is a corticosteroid.

13. The method of claim 11, wherein the non-steroidal anti-inflammatory agent is ibuprofen.

14. The method of claim 9, wherein the vasoconstrictor is betamethasone dipropionate.

15. A topical composition for treating alopecia comprising an effective amount of hydralazine in a pharmaceutically acceptable preparation.

16. The composition of claim 15, wherein the hydralazine is present in the preparation in an amount of from about 0.5% to about 4%.

17. The composition of claim 16, wherein the hydralazine is present in the preparation in an amount of about 2%.

18. The composition of claim 15, wherein the hydralazine is in a pharmaceutically acceptable aqueous preparation further comprising a glycol and an alcohol.

19. The composition of claim 18, wherein the glycol is ethylene glycol or propylene glycol

20. The composition of claim 18, wherein the alcohol is a monohydroxy alcohol selected from the group consisting of methanol, methanol, ethanol, isopropanol, and n-propanol.

21. The composition of claim 18, wherein the hydralazine is hydralazine HCl and the pharmaceutically acceptable aqueous preparation comprises about 25% propylene glycol and about 50% ethanol.

22. The composition of claim 15, wherein the composition is suitable for twice daily topical administration.

23. The composition of claim 15, wherein the composition further comprises an agent selected from the group consisting of a type II 5α-reductase, an anti-inflammatory agent, and a vasoconstrictor.

24. The composition of claim 23, wherein the a type II 5α-reductase is finasteride.

25. The method of claim 23, wherein the anti-inflammatory agent is a steroidal or non-steroidal anti-inflammatory agent.

26. The method of claim 25, wherein the steroidal anti-inflammatory agent is a corticosteroid.

27. The method of claim 25, wherein the non-steroidal anti-inflammatory agent is ibuprofen.

28. The method of claim 23, wherein the vasoconstrictor is betamethasone dipropionate.
